(19) United States
(12) Patent Application Publication
(10) Pub. No.: US 2004/0073282 A1
(43) Pub. Date: Apr. 15, 2004

(54) Distally-narrowed vascular grafts and methods of using same for making artery-to-vein and artery-to-artery connections

(76) Inventor: Paul Stanish, Crown Point, IN (US)

Correspondence Address: Woodard Emhardt Naughton, Moriarty & Mcnett, Suite 3700, 111 Monument Circle, Indianapolis, IN, 46204, US

(21) Appl. No.: 10/344,221

(22) PCT Filed: Aug. 6, 2001
(86) PCT No.: PCT/US01/24610

(51) Int. Cl. A61F 2/06

(52) U.S. Cl. 623/1.3; 623/1.36; 606/155

(57) ABSTRACT

The invention provides vascular grafts and methods for implanting the same. In one aspect of the invention, there are provided vascular grafts that have a stepped down distal end. In another aspect, the invention provides vascular grafts having cuffs for attachment to a target blood vessel. In yet another aspect, the invention provides connectors for making end-to-end graft connections. The invention also provides methods for implanting vascular grafts that include inserting the distal end of a graft into the target blood vessel for positioning downstream of the incision site and methods that include shortening a graft by removing a medial portion of the graft and connecting the resulting graft pieces in an end-to-end manner.

### REFERENCE TO RELATED APPLICATIONS

This application claims the benefits of U.S. Provisional Application No. 60/224,392, filed Aug. 10, 2000 and entitled ARTERIOVENOUS GRAFTS AND METHODS OF IMPLANTING THE SAME; U.S. application Ser. No. 09/704,083, filed Nov. 1, 2000 and entitled ARTERIOVENOUS GRAFTS AND METHODS OF IMPLANTING THE SAME; U.S. Provisional Application No. 60/297,088 filed Jun. 8, 2001 and entitled DISTALLY NARROWED VASCULAR GRAFTS AND METHODS OF USING SAME FOR MAKING ARTERY-TO-VEIN AND ARTERY-TO-ARTERY CONNECTIONS and U.S. Provisional Application No. 60/297,226 filed Jun. 8, 2001 and entitled ARTERIOVENOUS GRAFTS AND RELATED DEVICES AND METHODS OF USING SAME, which are hereby incorporated by reference herein in their entirety.

### BACKGROUND OF THE INVENTION

The present invention relates to vascular grafts and methods for implanting the same. It relates in various aspects to a vascular graft that has a stepped down distal end, a vascular graft having a cuff that aids in the attachment of the graft to a target blood vessel, a connector device for making an end-to-end graft connection and methods relating thereto. In various embodiments of the invention, the vascular graft can be used for making an artery-to-vein connection (referred to herein as an arteriovenous graft) or for making an artery-to-artery connection.

As a background to vascular grafts used for making artery-to-vein connections, it has been reported that approximately 40,000 new patients per year begin hemodialysis. This number continues to rise annually at a rate of about 10 percent. It has also been reported that creation and maintenance of hemodialysis access accounted for approximately $800 million in physician and hospital expenditures in 1998. The term “access” identifies a location within a patient's circulatory system that is specifically configured to act as a site for supplying blood to a dialysis machine and for receiving blood returning from a dialysis machine. Preferred modes of access, if the patient's condition allows, are grafts or fistulas, each of which is a subcutaneous device in fluid communication with the patient's circulatory system into which needles can be repeatedly inserted for transferring blood to and from a dialysis machine during dialysis treatments.

The most common cause of a hospital admission for dialysis patients is an access-related problem, and the cost and frustration of access failure is expected to continue to rise until a solution to the current access problem is identified and instituted. Even in view of this great need, no major advances in hemodialysis access have occurred in the last 30 years. Hakim and Himmelfarb, in their article entitled “Hemodialysis Access Failure: A Call to Action,” concluded by saying, “as far as [hemodialysis] access, we can no longer continue doing the same thing (very little indeed), the same way . . . , and expect that the lives of our [end stage renal disease] patients will be better or that the cost of access care will diminish.” Hakim R, Himmelfarb J. Hemodialysis Access Failure: A Call to Action, Kidney International Vol 54, (1998), pp. 1029-1040. If the patency rate of an access could be doubled, hundreds of millions of dollars would be saved annually and could be in turn directed toward transplant programs or a cure for renal disease. In addition, dialysis patients, particularly patients suffering from end stage renal disease (“ESRD”), would then be able to enjoy more productive, healthy and happy lives.

A primary arteriovenous fistula (AVF) is the preferred and most cost-effective long-term access for hemodialysis patients. An AVF is an artificial direct connection between an artery and a vein. High blood flow through this connection causes the vein to become much larger and develop a thicker wall, more like an artery. The AVF thus provides a high blood-flow site for accessing the circulatory system for performing hemodialysis. For each dialysis, two large-bore needles (normally 14-16 gauge) are inserted through the dialysis patient's skin and into the AVF, one on the “arterial” end and the other on the “venous” end. When the tips of the needles are properly resting inside the access, a column of blood enters the end of tubing attached to each needle. Prior to beginning a dialysis treatment, a cap is removed from each tubing, thereby allowing blood to fill the tubing, and then a syringe of saline is injected through each tubing and needle. The two needles are then connected with rubber tubing to the inflow (arterial) and outflow (venous) lines of the dialysis machine, and dialysis is started.

Unfortunately, even with careful physical examination and/or the use of doppler ultrasound or venography to identify suitable veins, it has been reported that approximately 40-50% of patients do not have the vascular anatomy sufficient to create a primary AVF. In addition, many dialysis veterans, for whom the use of an AVF has previously failed, can no longer be considered candidates for a primary fistula.

The next option for long-term access is the placement of an arteriovenous graft (“AVG”). An AVG is a length of plastic tube, usually made of porous polytetrafluoroethylene (“PTFE”), which is surgically placed under the skin, fluidly connecting an artery and a vein. Once a graft is placed, a dialysis machine can be fluidly connected to the patient's circulatory system by inserting needles into the graft and connecting the needles to the dialysis machine with tubing as described generally above in connection with an AVF. The phrase “long-term access,” however, is a misnomer when discussing an AVG, because reported patency rates are very low, with one-year patency rates of only 40% frequently reported. Even with graft surveillance and aggressive prophylactic measures to prevent thrombosis, primary and secondary patency rates remain low; and the additional costs incurred are also thought to be unreasonably high.

Although many factors have a role in the limited durability of PTFE grafts, enemy number one is the progressive development of neointimal hyperplasia (NIH) with venous anastomotic narrowing and subsequent graft thrombosis. Thrombosed grafts can be declotted surgically or percutaneously, or elaborate atherectomy devices, angioplasties, or stenting procedures can be utilized; but still, the NIH will predictably recur. A revision of the graft with a more proximal anastomosis can also be completed but this too has been found to be very susceptible to failure. There is a great need to improve the patency rates of AVGs to decrease cost and improve patient quality of life.

Neointimal hyperplasia is thought to occur because of many factors, including, for example, injury to endothelium with release of growth factors, turbulent flow at the anastomotic site, vibratory forces on the anastomosis, shear forces, uremia, and other hypothetical etiologies. A multitude of studies show the persistent and consistent development of NIH at the venous anastomotic site.

Physicians have used stepped and tapered grafts, which typically have a gradually increasing diameter along a portion of the graft or along the entire length of the graft from the arterial end to the venous end, and which terminate at the venous end with an opening at a point having the greatest diameter of the graft. An example of a tapered graft having a continuously increasing diameter is set forth in FIG. 1, wherein the diameter of the lumen at venous end 1 is larger than the diameter at the arterial end 2.

A general belief that has become widespread among medical practitioners is that the development of NIH could be prevented or delayed by further increasing the size of the venous anastomosis. In an attempt to improve AVG durability, “hooded grafts” with a larger venous anastomosis have been utilized. A representative example of a prior art hooded graft (Venaflo, Impra Company) is set forth in FIG. 2, wherein the venous end 3 has a “hood” configuration. However, no significant improvement has been reported resulting from the use of such a hooded graft. Indeed, the Venaflo graft has been found in some cases to occlude sooner than a standard stepped graft. Furthermore, attempts to salvage the graft by surgical or nonsurgical thrombectomy have been found to be more difficult to accomplish and have been found to result in comparatively shorter secondary patency rates.

Similar difficulties can arise when a graft is used to shunt blood from one artery to another by fluidly connecting two arteries or to bypass a portion of an artery by fluidly connecting the ends of a graft to a single artery at two different locations. Such a graft may be used, for example, when a portion of an artery has become blocked or damaged or when it becomes necessary or desirable to cause blood flow to bypass a portion of an artery due to other blood flow impairments or for other reasons. It is generally understood, however, that placement of an arterial bypass graft results in disruption of natural blood flow in an artery, and is therefore also associated with a significant risk of neointimal hyperplasia (NIH), distal anastomotic narrowing and subsequent graft thrombosis.

In view of the above, there is a great need for alternative modes of treatment and care of hemodialysis patients and treatment and care of other patients requiring placement of a vascular graft, such as an arterial bypass graft. In particular, a great need exists for new hemodialysis access grafts and for new arterial bypass grafts, and methods for surgically placing the same. These needs are addressed by the present invention.

### SUMMARY OF THE INVENTION

The present invention addresses problems associated with hemodialysis access grafts and other vascular grafts known in the prior art by providing novel grafts and methods for surgically placing the same. The embodiments of the invention exhibit a variety of excellent features.

One form of the present invention is a unique vascular graft having a stepped down distal end. The distal end portion can include a section of generally constant diameter that is adjacent a stepped section, which is in turn adjacent a section of a larger generally constant diameter. The stepped section has a decreasing diameter in the direction of blood flow through the graft, i.e., in a direction toward the distal end. Alternatively, the stepped section having a decreasing diameter can extend to the distal end from an adjacent section of larger diameter. The stepped section can vary in length and slope, and can provide a generally linear decrease in diameter or a nonlinear decrease. Another form of the invention is a vascular graft that also has a stepped proximal end. In certain embodiments, an inventive graft includes externally detectable markings, such as, for example, radiopaque markings, which facilitate subsequent identification and location of the graft and thereby facilitate surveillance or interventional therapy of the graft.

Another form is a unique vascular graft having a cuff positioned a preselected distance from the distal end for attachment to the wall of a target blood vessel. The cuff defines a groove for receiving a purse-string suture in the vessel wall. The cuff can be a single unit affixed to the exterior surface of the graft or can include a plurality of units. In one embodiment, the cuff comprises two rings about the circumference of the graft at a preselected position that are spaced from one another to provide a groove therebetween for receiving the suture and vessel wall. In another embodiment, at least a portion of the cuff, such as, for example, one or both of the rings includes an externally detectable marking, such as, for example, a radiopaque marking, to facilitate subsequent identification and location of the anastomotic site and thereby facilitate surveillance or interventional therapy that a graft might require.

Another form of the invention includes a method for implanting a vascular graft that includes making an incision in the wall of a target vessel, inserting the distal end of an inventive graft into the vessel such that the distal end is positioned downstream of the incision site, and securing the graft to the vessel wall. In an embodiment in which a cuffed graft is used, the securing includes making a purse-string suture that engages the groove of the cuff. In certain embodiments, the method also includes removing a medial portion of the graft to thereby shorten the graft. For example, a medial portion of the graft can be removed by cutting the graft at two predetermined points, separated by a distance equal to the length desired to be removed, to thereby provide two cut ends, and connecting the cut ends to one another to provide a shortened graft.

In another form of the invention, there is provided a connector device that finds advantageous use in aiding the connection of ends of a plurality of conduits, such as, for example, blood vessels, grafts and the like. In an embodiment for connecting two conduits, for example, the connector includes a first segment configured for insertion into the end of one conduit and a second segment configured for insertion into the end of the other conduit. In certain embodiments, the device also includes a middle segment for maintaining the cut ends in a spaced apart relationship.

It is one object of the invention to provide novel vascular grafts and methods for implanting the same that provide an alternative to the unsatisfactory grafts and methods of the prior art.

It is another object of the invention to provide methods and devices for shortening a vascular graft by removing a medial portion from the graft.

Further forms, embodiments, objects, advantages, benefits, aspects and features of the present invention will be apparent from the drawings and detailed description herein.

### BRIEF DESCRIPTION OF THE FIGURES

Although the characteristic features of this invention will be particularly pointed out in the claims, the invention itself, and the manner in which it may be made and used, may be better understood by referring to the following descriptions taken in connection with the accompanying figures forming a part hereof.

FIG. 1 depicts a prior art standard tapered graft having increasing diameter from proximal end 2 to distal end 1.

FIG. 2 depicts a prior art hooded graft.

FIG. 3 is a side view of an embodiment of a vascular graft in accordance with the invention.

FIG. 4 is a side view of another embodiment of a vascular graft in accordance with the invention.

FIG. 5 is a side view of another embodiment of a vascular graft in accordance with the invention.

FIG. 6 is a side view of another embodiment of a vascular graft in accordance with the invention.

FIG. 7 is a partial diagrammatic cutaway side view of an inventive graft after implantation of the graft.

FIG. 8 is a side view of an embodiment of a cuff in accordance with the invention.

FIG. 9 is a side view of another embodiment of a vascular graft in accordance with the invention.

FIG. 10 is a side view of another embodiment of a vascular graft in accordance with the invention.

FIG. 11 is a diagrammatic partial cutaway side view of an inventive graft after implantation of the graft.

FIG. 12 is a perspective view of an embodiment of a connector device in accordance with the invention.

FIG. 13 is a perspective view of the connector device of FIG. 12, also showing graft pieces being connected.

FIG. 14 is a perspective view of the connector device of FIG. 12, depicting the device positioned for connection of two graft pieces.

FIG. 15 is a perspective view of another embodiment of a connector device in accordance with the invention.

FIG. 16 is a perspective view of the connector device of FIG. 15, depicting the device in use for connection of graft pieces.

FIG. 17 is a perspective view of another embodiment of a connector device in accordance with the invention.

FIG. 18 is a perspective view of another embodiment of a connector device in accordance with the invention.

FIG. 19 is a perspective view of another embodiment of a connector device in accordance with the invention.

FIG. 20 is a perspective view of another embodiment of a connector device in accordance with the invention.

FIG. 21 is a perspective view of the connector device of FIG. 12, depicting the device positioned for connection of two graft pieces, and also showing the distal end of the connected graft positioned in a blood vessel.

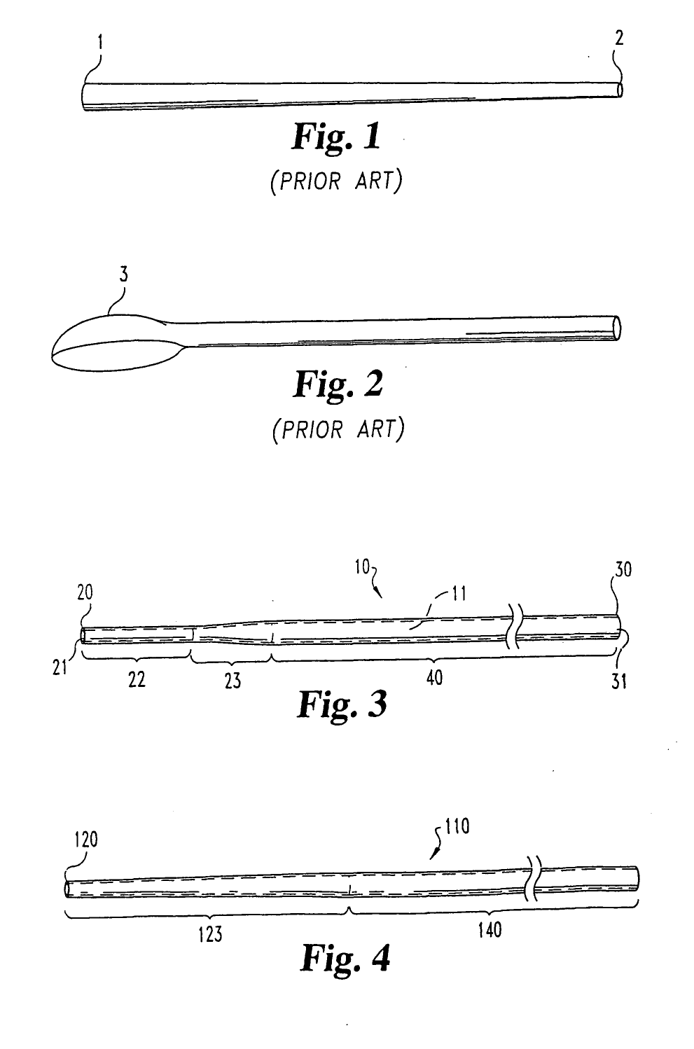

### DESCRIPTION OF PREFERRED EMBODIMENTS

For the purposes of promoting an understanding of the principles of the invention, reference will now be made to preferred embodiments and specific language will be used to describe the same. It will nevertheless be understood that no limitation of the scope of the invention is thereby intended, such alternations and further modifications in the invention, and such further applications of the principles of the invention as described herein being contemplated as would normally occur to one skilled in the art to which the invention pertains.

The present invention provides in one aspect a vascular graft prosthesis (hereinafter “graft”) comprising a generally tubular conduit defining a lumen for passage of blood and having a tapered or stepped-down distal end. The distal end is configured for insertion inside a target blood vessel through an incision to direct blood flow away from the incision site and to provide for a more laminar flow of blood from distal end of the graft into the vein or artery to thereby decrease turbulent flow at the anastomotic site.

Referring to FIG. 3, which illustrates one embodiment of the invention, graft 10 has a first end 20 adapted for insertion into a blood-receiving vessel site (referred to herein as the “distal end” of the graft) and a second end 30 adapted for attachment to an artery site selected to supply blood to the graft (referred to herein as the “proximal end” of the graft). It is understood by a person of ordinary skill in the art that blood flows unidirectionally through a vasular graft. As used herein, the terms “proximal end” and “distal end” are consistently used to identify the ends of a graft in relation to the direction of the flow of blood therein. As these terms are used herein, the flow of blood is in the direction from the proximal end to the distal end. In other words, blood passes from an artery site into the proximal end of a graft, flows through the graft toward the distal end of the graft, and then flows into a blood-receiving vessel such as, for example, a vein, a second artery or a downstream location of the same artery to which the proximal end is fluidly attached.

Graft 10 is preferably made of a material having a generally uniform thickness, and graft 10 therefore defines a lumen 11 having generally the same shape as the external surface of the graft. In graft 10, distal end 20 defines orifice 21 and proximal end 30 defines orifice 31. Orifice 21 and the portion of the graft lumen defined by section 22 of graft 10 have a generally constant diameter that is less than the diameter of orifice 31 and the portion of the graft lumen defined by sectin 40 of graft 10. The portion of the graft lumen 11 defined by section 23, also referred to herein as a “stepped” section or a “taper” section, has a generally gradually decreasing diameter, also described herein as a generally continuous taper, from a longitudinal point adjacent sectin 40 to a longitudinal point adjacent section 22.

In one embodiment, the portion of graft lumen 11 defined by sectin 40 has a diameter at least about 20% greater than that defined by section 22. In another embodiment, the portion of graft lumen 11 defined by sectin 40 has a diameter at least about 30% greater than that defined by section 22. In another embodiment, the portion of graft lumen 11 defined by sectin 40 has a diameter at least about 40% greater than that defined by section 22. In another embodiment, the portion of graft lumen 11 defined by sectin 40 has a diameter at least about 50% greater than that defined by section 22.

In one embodiment, the portion of lumen 11 defined by section 22 has an inner diameter of no greater than about 6 millimeters. In another embodiment, the portion of lumen 11 defined by section 22 has an inner diameter of no greater than about 5 millimeters. In another embodiment, the portion of lumen 11 defined by section 22 has an inner diameter of from about 3 to about 6 millimeters and the portion of lumen 11 defined by sectin 40 has an inner diameter of from about 4 to about 8 millimeters. In another embodiment, the portion of lumen 11 defined by section 22 has an inner diameter of from about 3.5 to about 4.5 millimeters and the portion of lumen 11 defined by sectin 40 has an inner diameter of from about 5.5 to about 7.0 millimeters.

In one embodiment, section 23 extends for a relatively short length of graft 10, such as, for example, less than about 2 centimeters. In such an embodiment, section 23 is preferably positioned from about 1 to about 10 centimeters from distal end 20. In another embodiment, section 23 is positioned from about 1 to about 8 centimeters from distal end 20, and in yet another embodiment, section 23 is positioned from about 1 to about 6 centimeters from distal end 20. In another embodiment, section 23 extends along a greater length of the graft, such as, for example, greater than about 2 centimeters, thereby providing a more gradual taper. In one embodiment, section 23 extends along a length of the graft of from about 1 to about 10 centimeters.

In another embodiment, depicted in FIG. 4, graft 110 does not include a section corresponding to section 22 in FIG. 3, and tapered section 123 generally continuously tapers from section 140 of greater diameter to distal end 120. In one embodiment, section 123 extends along the longitudinal axis of graft 110 from a longitudinal point at least about 2 centimeters from distal end 120. In another embodiment, section 123 extends from a longitudinal point at least about 4 centimeters from distal end 120. In another embodiment, section 123 extends from a longitudinal point from about 2 centimeters to about 15 centimeters from distal end 120. In another embodiment, section 123 extends from a longitudinal point from about 4 centimeters to about 15 centimeters from distal end 120. In another embodiment, section 123 extends from a longitudinal point from about 2 centimeters to about 10 centimeters from distal end 120. In another embodiment, section 123 extends from a longitudinal point from about 4 centimeters to about 10 centimeters from distal end 120.

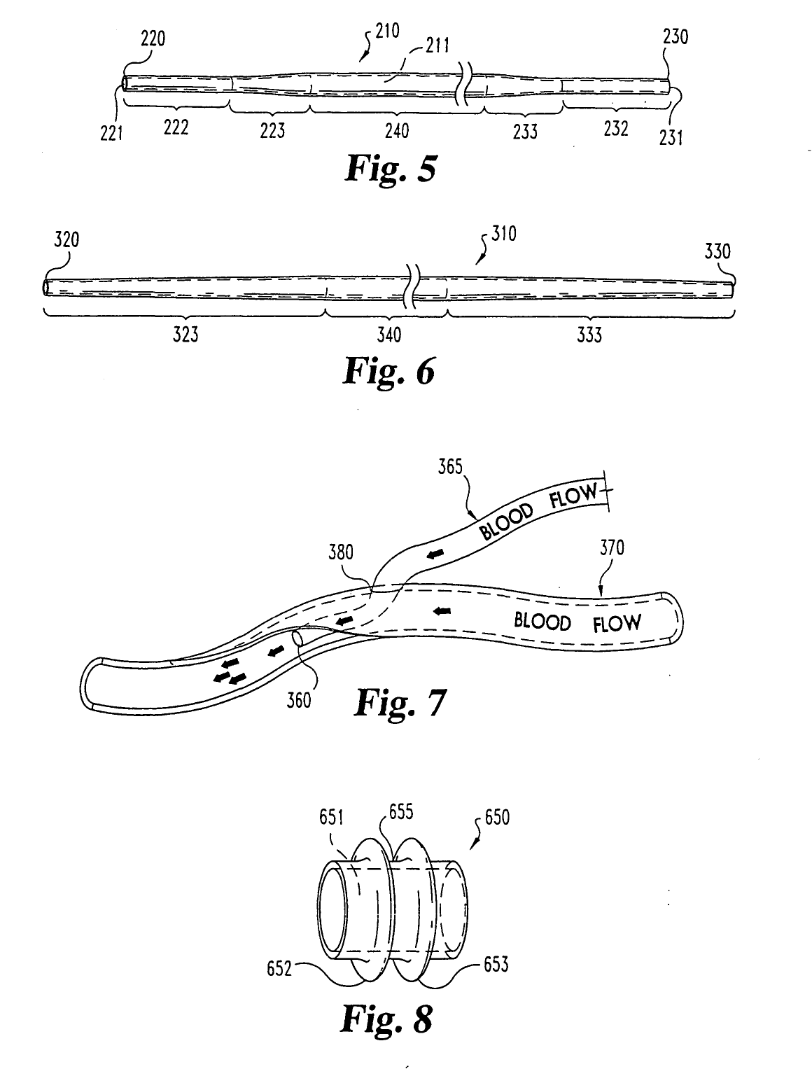

Referring now to FIG. 5, in another embodiment of the invention, in addition to having a stepped-down distal end portion, graft 210 also has a stepped-down proximal end portion. Graft 210 has a first end 220 adapted for insertion into a blood-receiving vessel site and a second end 230 adapted for attachment to an artery site selected to supply blood to the graft. In graft 210, distal end 220 defines orifice 221 and proximal end 230 defines orifice 231. Orifice 221 and the portion of the graft lumen defined by section 222 of graft 210 have a generally constant diameter that is less than the diameter of the portion of the graft lumen defined by section 240 of graft 210. In addition, orifice 231 and the portion of the graft lumen defined by section 232 of graft 210 have a generally constant diameter that is less than the diameter of the portion of the graft lumen defined by section 240 of graft 210. The portion of the graft lumen 211 defined by section 223, also referred to herein as a “stepped” section or a “taper” section, has a generally gradually decreasing diameter from the longitudinal point adjacent section 240 to the longitudinal point adjacent section 222, also described herein as a generally continuous taper. Similarly, the portion of the graft lumen 211 defined by section 233, also referred to herein as a “stepped” section or a “taper” section, has a generally evenly increasing diameter from the longitudinal point adjacent section 232 to the longitudinal point adjacent section 240.

In one embodiment, the portion of graft lumen 211 defined by section 240 has a diameter at least about 20% greater than that defined by section 222 or section 232. In another embodiment, the portion of graft lumen 211 defined by section 240 has a diameter at least about 30% greater than that defined by section 222 or section 232. In another embodiment, the portion of graft lumen 211 defined by section 240 has a diameter at least about 40% greater than that defined by section 222 or section 232. In another embodiment, the portion of graft lumen 211 defined by section 240 has a diameter at least about 50% greater than that defined by section 222 or section 232. In another embodiment, the portion of graft lumen 211 defined by section 240 has a length that is at least about 50% of the length of the graft. In another embodiment, the portion of graft lumen 211 defined by section 240 has a length that is at least about 60% of the length of the graft. In another embodiment, the portion of graft lumen 211 defined by section 240 has a length that is at least about 70% of the length of the graft. In another embodiment, the portion of graft lumen 211 defined by section 240 has a length that is at least about 80% of the length of the graft. In another embodiment, the portion of graft lumen 211 defined by section 240 has a length that is at least about 90% of the length of the graft. In another embodiment, the portion of graft lumen 211 defined by section 240 has a length that is at least about 95% of the length of the graft.

In one embodiment, the portion of lumen 211 defined by section 222 has an inner diameter of no greater than about 6 millimeters. In another embodiment, the portion of lumen 211 defined by section 222 has an inner diameter of no greater than about 5 millimeters. In another embodiment, the portion of lumen 211 defined by section 232 has an inner diameter of no greater than about 6 millimeters. In another embodiment, the portion of lumen 211 defined by section 232 has an inner diameter of no greater than about 5 millimeters. In another embodiment, the portion or portions of lumen 211 defined by one or both of section 222 and section 232 have an inner diameter of from about 3.5 to about 6 millimeters and the portion of lumen 211 defined by section 240 has an inner diameter of from about 4 to about 8 millimeters. In another embodiment, the portion or portions of lumen 211 defined by one or both of section 222 and section 232 have an inner diameter of from about 3.5 to about 4.5 millimeters and the portion of lumen 211 defined by section 240 has an inner diameter of from about 5.5 to about 7.5 millimeters. In another embodiment, the portion or portions of lumen 211 defined by one or both of section 222 and section 232 have an inner diameter of from about 3.5 to about 4.5 millimeters and the portion of lumen 211 defined by section 240 has an inner diameter of from about 5.5 to about 6.5 millimeters. In another embodiment, the portion or portions of lumen 211 defined by one or both of section 222 and section 232 have an inner diameter of about 4 millimeters and the portion of lumen 211 defined by section 240 has an inner diameter of about 6 millimeters.

Sections 223 and 233 can extend for a relatively short length of graft 210, such as, for example, less than about 2 centimeters each. In such an embodiment, section 223 is preferably positioned from about 1 to about 10 centimeters from distal end 220 and section 233 is preferably positioned from about 1 to about 10 centimeters from proximal end 230. In another embodiment, section 223 is positioned from about 1 to about 8 centimeters from distal end 220, and section 233 is positioned from about 1 to about 8 centimeters from proximal end 230. In yet another embodiment, section 223 is positioned from about 1 to about 6 centimeters from distal end 220 and section 233 is positioned from about 1 to about 6 centimeters from proximal end 230. It is to be understood that sections 223 and 233 can have the same length or different lengths. Alternatively, sections 223 and 233 can each extend along a greater length of the graft, such as, for example, greater than about 2 centimeters, thereby providing a more gradual taper. In one embodiment, sections 223 and 233 each extend along a length of the graft of from about 2 to about 10 centimeters.

In another embodiment, depicted in FIG. 6, graft 310 does not include sections corresponding to sections 222 and 232 in FIG. 5. Rather, tapered section 323 generally continuously tapers from section 340 of greater diameter to distal end 320 and tapered section 333 features a generally continuously increasing diameter from proximal end 330 to section 340 of greater diameter. One or both of sections 323 and 333 preferably extends along the longitudinal axis of graft 310 to a longitudinal point at least about 2 centimeters from respective ends 320 and 330. In another embodiment, one or both of sections 323 and 333 extends to a longitudinal point at least about 4 centimeters from respective ends 320 and 330. In another embodiment, one or both of sections 323 and 333 extends to a longitudinal point from about 2 centimeters to about 15 centimeters from respective ends 320 and 330. In another embodiment, one or both of sections 323 and 333 extends to a longitudinal point from about 4 centimeters to about 15 centimeters from respective ends 320 and 330. In another embodiment, one or both of sections 323 and 333 extends to a longitudinal point from about 2 centimeters to about 10 centimeters from respective ends 320 and 330. In another embodiment, one or both of sections 323 and 333 extends to a longitudinal point from about 4 centimeters to about 10 centimeters from respective ends 320 and 330.

Grafts described herein are preferably made from polytetrafluoroethylene (“PTFE”); however, it is not intended that the invention be limited by the material from which the graft is made. It is contemplated that the graft can advantageously be made from a wide variety of biocompatible materials, now known or later developed, having acceptable physical characteristics of flexibility and the like.

Also provided by the present invention are methods for surgically implanting inventive grafts. To place a graft in accordance with the invention, an incision is made in a blood vessel at a location selected to receive blood from the graft, the distal end of the graft is introduced into the interior of the blood vessel at the incision site and placed a predetermined distance downstream from the incision, and the graft is sealingly secured to the wall of the blood vessel at the incision site. A representative configuration of the graft in relation to the target blood vessel site after placement is depicted in FIG. 7, in which the distal end 360 of graft 365 is positioned within the target blood vessel 370 at a location downstream from the incision site 380 relative to the natural, flow of blood in the blood vessel 370.

In one embodiment, attachment of the distal end portion of an inventive graft to a target blood vessel site is achieved by (1) making an incision in the wall of a target blood vessel at a predetermined site; (2) inserting the distal end of an inventive graft through the incision into the blood vessel such that the first end passes to a point downstream of the incision; (3) securing the graft to the blood vessel at the incision site; and (4) anastomosing the proximal end to a preselected artery site. In one embodiment, the distal end of the graft can be advantageously secured to the target blood vessel using a purse-string suture. In one manner of practicing the invention, the purse-string suture can be inserted in a wall of the target blood vessel prior to making the incision. In another embodiment, the inserting includes inserting the distal end of an inventive graft through the incision into the blood vessel such that the distal end passes to a point at least about 1 centimeter downstream of the incision. In another embodiment, the inserting includes inserting the distal end of an inventive graft through the incision into the blood vessel such that the distal end passes to a point from about 1 to about 4 centimeters downstream of the incision. In another embodiment, the inserting includes inserting the distal end of an inventive graft through the incision into the blood vessel such that the distal end passes to a point from about 1 to about 3 centimeters downstream of the incision. Inserting the distal end of the graft into the blood vessel directs blood flow away from the incision site and therefore decreases the violent flow of blood at the anastomotic site.

One advantage of the invention is that the distal end of an inventive graft can be placed in blood-receiving blood vessels having a wide variety of sizes, including blood vessels that were previously thought to be unsuitable due to size limitations. In one manner of practicing the invention, the blood-receiving blood vessel at the target attachment site selected for anastomosis has a diameter of no greater than about 1.5 centimeters. In another embodiment, the selected blood-receiving blood vessel at the target attachment site has a diameter of from about 0.4 centimeters to about 1.5 centimeters. In another embodiment, the selected blood-receiving blood vessel at the target attachment site has a diameter of no greater than about 1.4 centimeters. In another embodiment, the selected blood-receiving blood vessel at the target attachment site has a diameter of no greater than about 1.3 centimeters. In another embodiment, the selected blood-receiving blood vessel at the target attachment site has a diameter of no greater than about 2 centimeters. It is also contemplated that the invention can be used for placement of an inventive graft in a large blood vessel, such as a blood vessel in a patient's leg. As such, in another embodiment, the selected blood-receiving blood vessel at the target attachment site has a diameter of up to about 3 centimeters.

In another aspect, the invention provides a graft that includes a cuff positioned a desired distance from the distal end of the graft and adapted to receive a purse-string suture at the incision site during the surgical placement of the graft, thereby isolating injured endothelium at the site of the incision from the intravascular portion of the anastomosis. In this regard, in one aspect of the invention, a graft is provided that includes a cuff affixed to the outer surface of the graft, the cuff defining a groove configured to receive a purse-string suture for anastomosis. It is to be understood that the present invention contemplates the use of such cuffs in connection with novel grafts described herein having a tapered or stepped down distal end as well as standard grafts, i.e., grafts that are not tapered at the distal end, and other grafts.

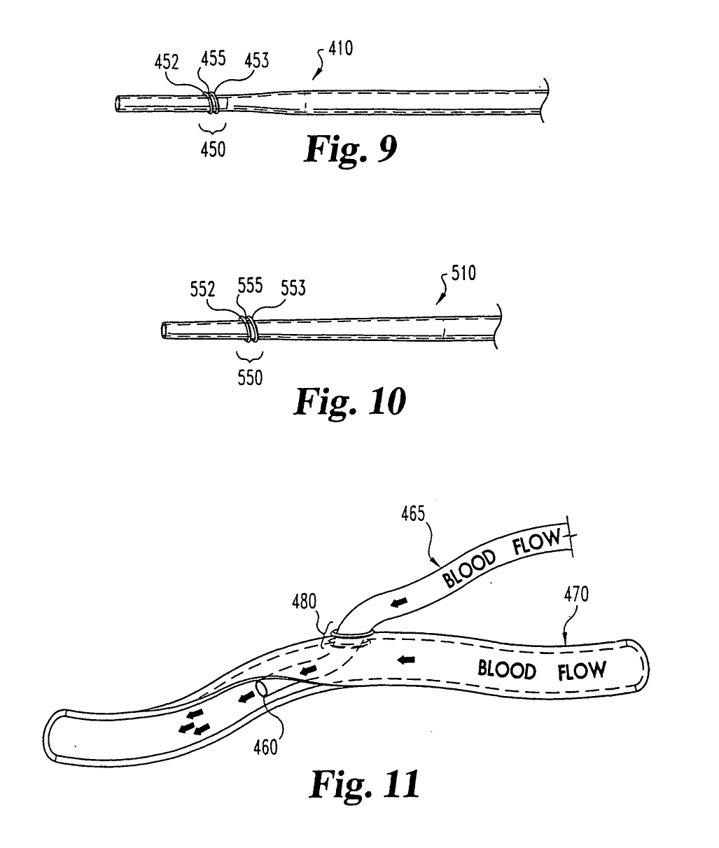

In one embodiment, the cuff is configured such that the groove lies on a plane that is generally perpendicular to a longitudinal axis of the graft. It is readily understood that with this arrangement, when the graft is sutured to a blood vessel wall, the graft will pass through the blood vessel wall generally at a 90 degree angle to the vein. In another embodiment, the cuff is configured such that the groove lies on a plane that is at an angle to a longitudinal axis of the graft, as shown in FIGS. 9 and 10. In one preferred embodiment, the groove lies on a plane that is at an angle of about 45 degrees to the longitudinal axis of the graft; however, the invention contemplates a wide variety of angles.

In one embodiment, the cuff includes a single structure defining a groove. For example, FIG. 8 depicts a cuff 650 configured for attachment to the external surface of a graft. Cuff 650 defines a lumen 651 that is sized to engage the external surface of a graft at a desired location. Cuff 650 also includes a first ridge 652 and a second ridge 653 extending around cuff 650 and forming a groove 655 therebetween. Cuff 650 can be attached to a graft at a desired location using a biocompatible adhesive or can be integrally formed to a graft.

In another embodiment, the cuff can include two ring structures extending around a graft and defining a groove therebetween. Referring now to FIGS. 9 and 10, grafts 410, 510 include cuffs 450, 550 affixed or integrally molded to the outer surface of grafts 410, 510, respectively. Cuffs 450, 550 each include two ring structures 452, 453, 552, 553 and each define a groove, 455, 555 configured to receive a purse-string suture for a venous or arterial anastomosis.

In one embodiment, the groove is positioned at least about 1 centimeter from the distal end. In another embodiment, the groove is positioned from about 1 centimeter to about 5 centimeters from the distal end. In another embodiment, the groove is positioned from about 1 to about 3 centimeters from the distal end. It is readily understood that the distance from the distal end affects the length of the graft that resides inside the target blood vessel after completion of a graft implantation procedure. In one embodiment, for example, a cuff is positioned such that the groove defined by the cuff is positioned from about 1 to about 3 centimeters from the distal end of the graft. In another embodiment, a cuff is positioned such that the groove defined by the cuff is positioned from about 1.5 to about 2.5 centimeters from the distal end of the graft. In another embodiment, wherein the cuff includes two ring structures as shown in FIGS. 9 and 10, one ring is positioned about 1.75 centimeters from the distal end of the graft and another ring is positioned about 2.25 centimeters from the distal end, defining the groove therebetween.

In another embodiment of the invention, at least a portion of a graft is coated and/or impregnated with an antithrombogenic material. In one embodiment, a surface of the graft that comes into contact with blood is coated and/or impregnated with the antithrombogenic material. The presence of an antithrombogenic on a surface of the graft has the advantageous effect of deterring blood coming into contact with the surface from clotting and/or adhering to the surface. Surfaces that can advantageously be coated or impregnated with an antithrombogenic material in accordance with certain embodiments of the invention include the surface of the graft lumen, the external surface of the distal end portion to be inserted into the target blood-receiving vessel (i.e., for a graft having a cuff, the portion of the graft between the cuff and the distal end), and, when a cuff is present, the external surface of the portion of the cuff that is to be placed within the lumen of the target vessel (i.e., the portion of the cuff nearest the distal end of the graft). Suitable antithrombogenic materials that can be used to coat and/or impregnate a graft surface include, for example, carbon, heparin and silicone.

In another embodiment, at least a portion of the cuff includes an externally detectable marking, such as, for example, a radiopaque marking. The presence of such a marking makes the location of the cuff determinable externally using radiation or other means, and thereby allows a determination, based upon the relative position of the cuff on the graft, of the location where the diameter of the graft changes and/or where the graft enters the blood vessel. This knowledge facilitates post-placement surveillance or interventional therapy that a graft might require. In certain preferred embodiments, one or both of the cuff's ridges or rings comprise externally detectable markings. In another embodiment, one or both of a graft's ends comprise an externally detectable marking to thereby facilitate subsequent methods for externally identifying the position of one or more end of the graft. In one preferred embodiment, the venous end is marked with an externally detectable marking.

As used herein, the term “externally detectable” is intended to refer to a property of a material whereby the material is imageable with medical imaging equipment, including fluoroscopy equipment, x-ray equipment, CT equipment, MRI equipment, ultrasonic equipment and other forms of remote imaging. A wide variety of medical imaging equipment is available commercially. As used herein, the term “externally detectable” encompasses radiopaque materials as well as materials that can be detected by medical imaging equipment due to its different density than surrounding materials or other property. For example, fluoroscopy can be utilized in the case of a radiopaque material. A radiopaque material selected for use in accordance with the invention can be a barium compound, such as, for example, barium sulfate, a metallic component, such as, for example, tantalum, or other radiopaque material. It is well within the purview of a person of ordinary skill in the art to select a suitable radiopaque material for use in connection with the present invention. Alternatively, MRI or ultrasonic examination can be used to detect a material different in density from the body and other surrounding materials.

The externally detectable marking can be provided, for example, by incorporating an inert externally detectable material into a paint or other coating applied to the cuff or a portion thereof or to a portion of the graft, such as a graft end; by making the cuff or a portion thereof or a portion of the graft from an externally detectable material; or by incorporating an externally detectable material into a polymer used to make the cuff or a portion thereof or a portion of the graft.

The invention also provides in certain aspects methods for non-invasively monitoring a patient having an inventive subcutaneous graft. In one embodiment, a method includes implanting in the patient an inventive graft featuring an externally detectable material and ascertaining the position and/or patency of the graft by examining the patient, using a non-invasive technique such as x-ray or fluoroscopic imaging, in the case of radiopaque materials, or MRI or ultrasonic scanning, in the case of differing density materials.

Also provided by the present invention are methods for implanting inventive grafts having cuffs as described herein. To place a graft having a cuff in accordance with the invention, an incision is made in a target blood vessel, the distal end of the graft is introduced into the interior of the blood vessel such that the cuff is positioned whereby the groove is positioned to receive the blood vessel wall at the incision site, and the blood vessel wall is secured to the graft. A representative configuration of a cuffed graft in relation to the target blood vessel after placement is depicted in FIG. 11, in which the distal end 460 of graft 465 is positioned within the target blood vessel 470 at a location downstream from the incision site relative to the natural flow of blood in the blood vessel 470. The cuff 480 is positioned so that the groove defined thereby receives the blood vessel wall at the incision site.

In one embodiment, anastomosis is achieved by (1) making an incision in the wall of a preselected target blood vessel; (2) inserting the distal end of a cuffed graft through the incision into the blood vessel such that the groove is positioned to receive the blood vessel wall and a suture; (3) securing the wall to the cuff; and (4) anastomosing the second end to a preselected target artery site. In one embodiment, the distal end of the graft can be advantageously secured to the blood-receiving blood vessel site using a purse-string suture. In one manner of practicing the invention, the purse-string suture can be inserted in a wall of the target blood vessel prior to making the incision and the securing includes securing the blood vessel wall to the cuff using the purse-string suture. In addition to directing the blood flow away from the incision site, reducing the forces of the graft on the anastomotic site, and making the blood flow more laminar, use of the present invention also significantly reduces the amount of injury to the blood vessel endothelium produced by the incision and suturing. Furthermore, constructing an anastomosis using a cuff and purse-string suture in accordance with the invention allows isolation of the incision itself (i.e., the cut edges of the blood vessel wall) from the interior of the blood vessel. Thus, blood in the graft and blood vessel does not come into contact with the incision site.

It is to be understood that the present invention also contemplates the use of cuffs in connection with standard grafts, i.e., grafts that are not tapered at the distal end. In addition, although embodiments including cuffs are described and shown herein, it is not intended that the invention be limited to grafts having cuffs, and the present invention in certain aspects as described above, provides configurations that do not include cuffs. Where a cuff is absent, the graft can be anastomosed to the blood-receiving blood vessel site using other techniques known in the art, and the graft optionally can include alternative structures for attachment to the blood vessel site as are known in the art.

Turning now to another advantageous feature of the present invention, it is readily understood by a person of ordinary skill in the art that graft placement commonly involves a modification to the length of a graft being placed. Arteriovenous grafts, for example, are typically manufactured to have an excess length that can be removed if necessary. One reason for this excess length is to provide a graft that can be used for patients of all sizes. Additionally, this provides a physician placing the graft a degree of latitude in selecting a suitable subcutaneous path along which the graft will lie from the target arterial site to the target venous site. During placement of an arteriovenous graft, therefore, the portion of the graft that is determined to be excess is removed, commonly by clipping the appropriate length from one end of the graft. In this regard, during placement of an arteriovenous graft, the graft can be tunneled subcutaneously from a point near the target venous anastomosis site to a point near the arterial anastomosis site (or vice versa). Excess length is then clipped from one or both ends if necessary to provide ends of the graft that are appropriately positioned for attachment to the respective vessels. Similar techniques are commonly used in procedures for placing artery-to-artery grafts.

It is apparent that the above-described procedure is not well suited for a graft placement procedure in which the graft being placed has a specifically desired configuration at each end, such as where a graft having a tapered or stepped distal end and a tapered or stepped proximal end in accordance with certain aspects of the invention is used. When placing this type of graft, it is typically not acceptable to reduce the length of the graft by simply clipping a predetermined length from one end of the graft. Therefore, to reduce the length of the graft, it is necessary to remove a predetermined length from a medial portion of the graft and to then connect the two cut ends in an end-to-end manner to provide a continuous graft conduit. In addition to the above, achieving an end-to-end connection of two pieces of graft can also be necessary when an existing arteriovenous graft, by-pass graft or the like must be revised. A revision of this type commonly involves making end-to-end connections of graft pieces. Sewing together two pieces of PTFE graft, however, for either a new or revised arteriovenous graft can be time consuming, cumbersome, and can lead to scarring and/or tissue ingrowth at the connection site. Thus, sewing graft pieces together in an end-to-end manner can result in stenosis which, if significant enough, can lead to thrombosis or failure of the graft. In addition, sewing together two ends of PTFE graft can lead to significant bleeding at the anastomotic site, further increasing the operative time and cost.

In another aspect of the invention, there is provided a connector device that finds advantageous use in aiding the connection of ends of graft pieces. The connector device of the invention finds particularly advantageous use when it is desired or necessary to reduce the length of an arteriovenous graft by removing a medial portion therefrom. In certain embodiments of the invention, after the medial portion is removed, the two cut ends of the graft are connected using an inventive connector device. It is of course not intended that the invention be limited to the connection of two cut ends of an arteriovenous catheter. An inventive connector device can be used to connect a wide variety of conduits in an end-to-end manner.

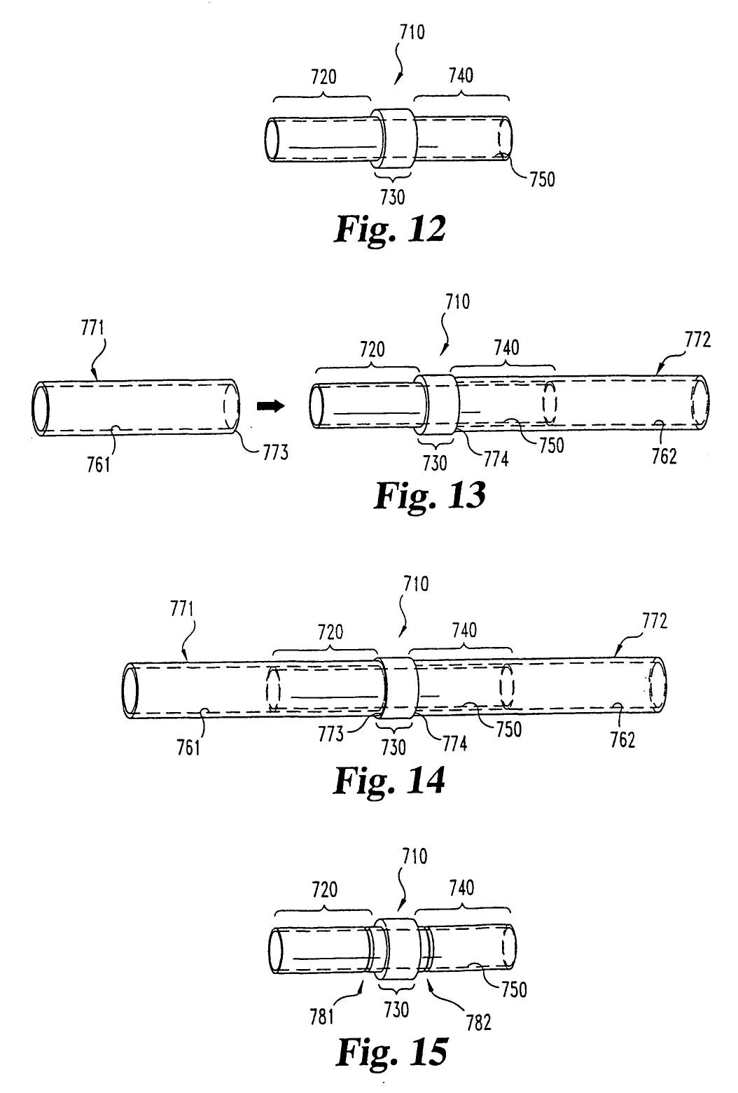

In one embodiment, depicted in FIG. 12, connector 710 is generally tubular and defines a lumen 750 configured for fluid communication with the lumens 761, 762 of the graft pieces or other conduits 771, 772 being connected, as shown in FIGS. 13 and 14. Connector 710 includes a first end segment 720 configured for insertion into the end 773 of a first graft piece or other conduit 771, a second end segment 740 configured for insertion into the end 774 of a second graft piece or other conduit 772 and a middle segment 730 of a greater outside diameter configured to maintain the graft piece ends 773, 774 in a spaced apart relationship.

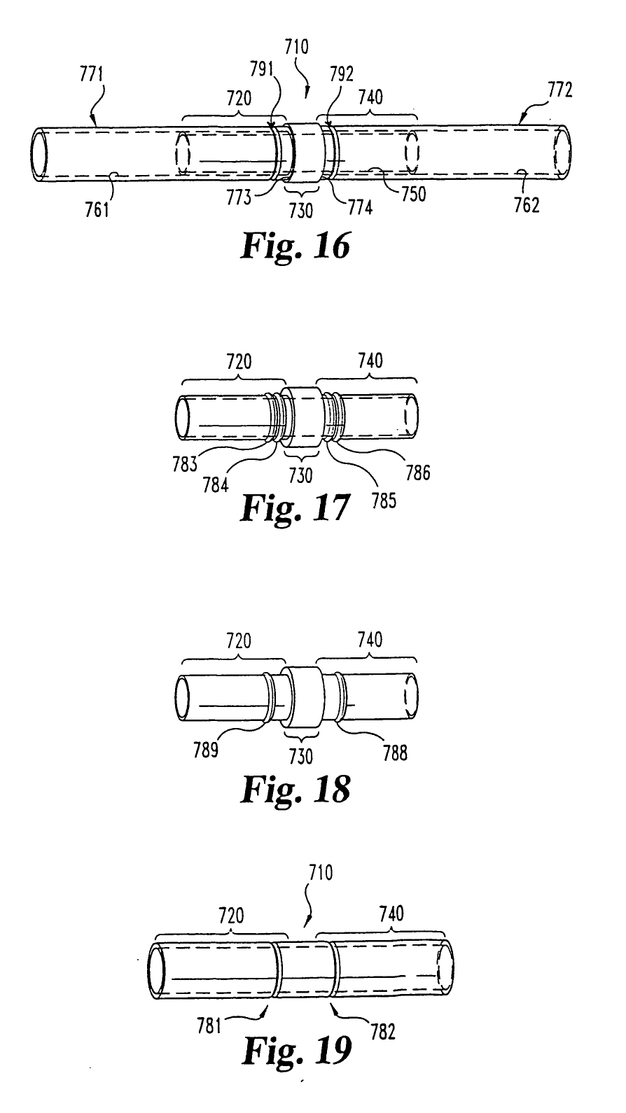

In other embodiments, connector 710 also comprises means for attaching graft pieces 771, 772 to connector 710. In one embodiment, for example, an adhesive composition is provided on the outside surfaces of first end segment 720 and second end segment 740 to hold connector 710 in place after it is placed into contact with graft pieces 771, 772. In another embodiment, depicted in FIG. 15, first end segment 720 and second end segment 740 each defines a groove 781, 782 for receiving a fastener such as, for example, a clamp, clip, tie filament or the like. FIG. 16 sets forth a representative example of a connector 710 in accordance with this embodiment connected to graft pieces 771, 772 by tie filaments 791, 792 positioned to engage grooves 781, 782. In another embodiment, depicted in FIG. 17, one or both of the grooves is formed between ridges or rings 783, 784, 785, 786 formed around segment 720 and/or 740. It is of course understood that the ridges or rings are configured to form a groove therebetween, but are preferably not so large as to make insertion of the segment into a graft end unacceptably difficult. In still another embodiment, depicted in FIG. 18, one or each of the first end segment 720 and the second end segment 740 includes a single ridge or ring 788, 789 configured to engage a fastener, such as, for example, a clamp, clip, tie filament or the like. A connector of this embodiment, can be secured to graft ends by inserting segments 720 and 740 into the graft ends such that the graft ends extend over ridges 788 and 789, and then positioning a selected fastener at a position adjacent the ridge to engage the ridge and thereby prevent slippage of the graft end from the end segment of the connector. It is of course understood that an inventive connector can alternatively be attached to graft pieces in other ways as would occur to a person of ordinary skill in the art.

In another embodiment of the invention, depicted in FIG. 19, no middle segment of greater outside diameter is present, and connector 710 includes a first segment 720 configured for insertion into the end of a first conduit and a second segment 740 configured for insertion into the end of a second conduit. Connector 710 defines grooves 781, 782 for receiving fasteners, such as, for example, a clamp, clip, tie filament or the like.

An inventive connector is preferably made from a relatively firm biomaterial. Firmness of an inventive connector can vary, but connectors are preferably sufficiently firm to withstand handling, insertion into graft pieces and the force of a filament, clip or other device used to secure the graft pieces to the connector. It is of course important that the connector also be sufficiently firm to prevent kinking or collapse at the connection site. It is also important that the material used to make a connector be non-biodegradable in the environment into which it is placed. For example, a connector used for connection of pieces of an arteriovenous graft or an arterial bypass graft must be made of a suitable material that is not degraded in the presence of blood under physiologic conditions. Selection of a suitable material is well within the purview of a person of ordinary skill in the art, and a variety of suitable materials are available commercially.

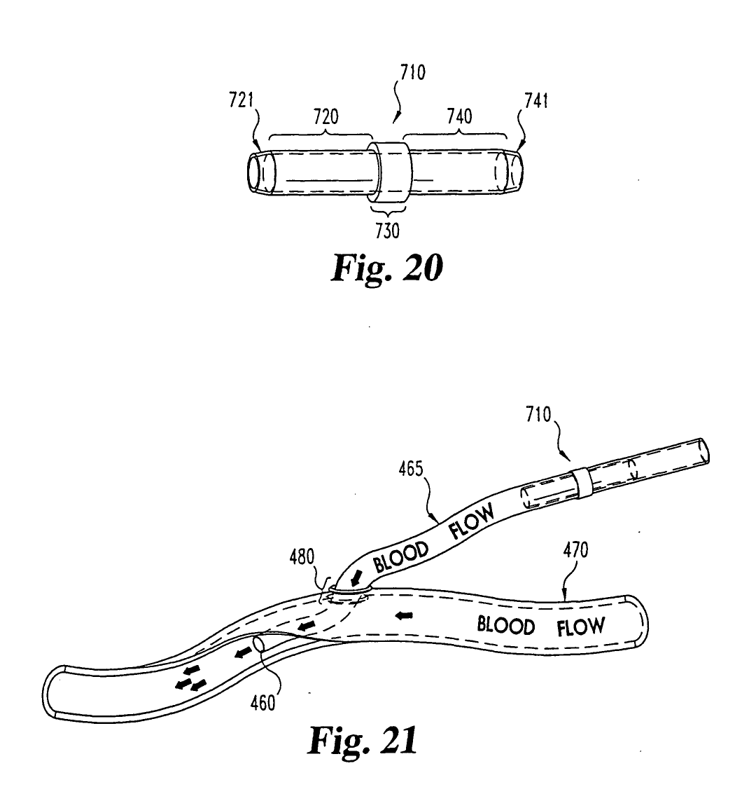

Inventive connectors are preferably sized to fit snugly into the ends of the conduits being connected. A snug fit helps prevent the formation of spaces or gaps between the connector and the respective graft ends, and helps facilitate the securing of the connector to the graft ends. It is therefore understood that connectors of different dimensions would be preferred when selected for use in connecting grafts of different dimensions. In certain embodiments of the invention, as depicted in FIG. 20, connector 710 includes tapered end portions 721 and 741 to facilitate insertion of the connector ends into the respective graft ends. It is of course preferred that the ends do not taper to a degree that would cause substantial turbulence in or obstruction to the flow of blood through the graft and connector after placement of the connector. It is well within the purview of a person of ordinary skill in the art to select taper dimensions that achieve the desired result of the invention. Insertion of an inventive connector into a graft end can also be facilitated if necessary by wetting the surfaces of the connector with saline to lubricate the connector prior to insertion. It is of course understood that alternative biocompatible fluids could also be used to wet and/or lubricate the connector and that a person of ordinary skill in the art is capable of selecting a suitable fluid to achieve this result.

It is expected that inventive connectors will often be used to connect graft pieces having a generally constant internal diameter. In one embodiment of the invention, the external diameter of the first end segment and the external diameter of the second end segment are approximately the same. Many arteriovenous grafts, for example, have internal diameters of from about 5 to about 8 mm. Therefore, in one preferred embodiment, the external diameter of the first end segment and the external diameter of the second end segment are from about 5 to about 8 mm. It is of course understood that inventive connectors can also be used to connect pieces of graft or other conduit having differing internal diameters. A connector used to connect such pieces will of course have a first end segment of a different external diameter than the second end segment. In one preferred embodiment, the diameter of the first end segment and the diameter of the second end segment are within about 0.5 mm of the internal diameters of the target graft pieces, respectively.

It is not necessary that inventive connector devices define lumens that are straight, as depicted in the drawings. Rather, in certain embodiments, inventive connectors are curved.

It is therefore apparent from the present specification that, in one embodiment of the invention, a vascular graft is provided that includes (1) a distal end adapted for placement in a blood-receiving blood vessel and defining a first orifice having a first diameter; (2) a proximal end adapted for attachment to an artery site; (3) a first tubular section between the distal and proximal ends, the first tubular section defining a portion of the lumen having a second, generally constant diameter greater than the first diameter; and (4) a first taper section between the first tubular section and the distal end, and having a generally gradually decreasing diameter along the first taper section. In another embodiment, a graft includes a second tubular section between the distal end and the first taper section, the second tubular section defining a portion of the lumen having a generally constant diameter corresponding to the first diameter.

In a further embodiment, the proximal end of a graft defines a second orifice having a third diameter less than the second diameter and the graft includes a second taper section between the proximal end and the first tubular section, the second taper section having a generally evenly increasing diameter. In still a further embodiment, a graft includes a third tubular section between the proximal end and the second taper section, wherein the third tubular section defines a portion of the lumen having a generally constant diameter corresponding to the third diameter.

In another embodiment, a cuff is affixed to the outer surface of the graft at least about 1 centimeter from the distal end, the cuff defining a groove configured to receive a purse-string suture for anastomosis. In yet a further embodiment, the groove lies generally on a plane that is at an angle of about 45 degrees to a longitudinal axis of the graft. The cuff in one embodiment includes a tubular member defining a lumen sized to engage an external surface of the graft; and first and second ridges extending around the member and defining the groove therebetween. In another embodiment, the cuff includes a first ring member affixed to the graft; and a second ring member affixed to the graft; wherein the first and second ring members define the groove therebetween.

In other embodiments, the cuff or other portion of the graft includes an externally detectable marker. In certain embodiments, the externally detectable marking is a radiopaque marking, while in other embodiments the externally detectable marking includes a material having a density that is different than the density of a patient's body. In certain embodiments, the cuff or other portion of the graft is coated with a paint or other coating composition including an externally detectable material

Another form of the invention is a method, including (1) making an incision in the wall of a preselected target blood vessel; (2) providing a vascular graft in accordance with the invention; (3) inserting the distal end of the graft through the incision into the blood vessel such that the distal end passes to a point downstream of the incision; (4) securing the graft to the blood vessel wall; and (5) anastomosing the proximal end of the graft to a preselected target artery site.

In another form of the invention, the graft is secured to the blood vessel using a purse-string suture. In another form of the invention, the purse-string suture is inserted in a wall of the target blood vessel prior to making the incision. In yet another form of the invention, the inserting includes inserting the distal end through the incision into the blood vessel such that the distal end passes to a point at least about 1 centimeter downstream of the incision. In another embodiment of the invention, the graft includes a cuff affixed to the outer surface of the graft at least about 1 centimeter from the distal end, the cuff defining a groove configured to receive a purse-string suture for anastomosis. In this embodiment, the inserting includes inserting the distal end of the graft through the incision into the blood vessel such that the groove is positioned to receive the blood vessel wall and the securing includes securing the blood vessel wall to the cuff. In still another manner of practicing the invention, a purse-string suture is inserted in a wall of the target blood vessel prior to making the incision and the securing includes drawing the purse-string suture into the groove.

In another form of the invention, there is provided a method that includes (1) anastomosing a first end of a vascular graft to a first blood vessel; (2) anastomosing a second end of the graft to a second blood vessel; (3) removing a medial portion of the graft by clipping the graft at two locations between the first end and the second end, thereby providing a first graft piece comprising the first end and a second graft piece comprising the second end; (4) providing a generally tubular connector device defining a lumen therethrough, the device having a first end segment configured for insertion into the lumen of the first graft piece; and a second end segment configured for insertion into the lumen of the second graft piece; and (5) connecting the first and second graft pieces to the first and second portions of the connector device. In certain embodiments, the connector device further includes a middle segment configured to maintain the graft piece ends in a spaced apart relationship. In other embodiments, each of the first and second end segments defines a groove or one or more rings for receiving a clip, clamp, tie filament or the like for attachment of the segments to the first and second graft pieces. In still other embodiments, the first and second end segments are coated with an adhesive composition to attach the segments to the first and second graft pieces. In yet other embodiments, the connecting is accomplished by: (1) inserting the first portion into the lumen of the first graft piece; (2) securing the second portion to the second graft piece; (3) inserting the second portion into the lumen of the second graft piece; and (4) securing the second portion to the second graft piece.

In another aspect of the invention, there is provided a generally tubular connector device defining a lumen therethrough, the device comprising: (1) a first end segment configured for insertion into the lumen of a first graft piece; (2) a second end segment configured for insertion into the lumen of a second graft piece; and (3) a middle segment configured to maintain the graft piece ends in a spaced apart relationship; wherein the first and second end segments are coated with an adhesive composition to attach the segments to the first and second graft pieces.

In still another aspect, the invention provides a generally tubular connector device defining a lumen therethrough, the device comprising: (1) a first end segment configured for insertion into the lumen of a first graft piece; and (2) a second end segment configured for insertion into the lumen of a second graft piece; wherein each of the first and second end segments defines a groove or one or more rings for receiving a clip, clamp, tie filament or the like for attachment of the segments to the first and second graft pieces.

In yet another aspect, the invention provides a generally tubular connector device defining a lumen therethrough, the device comprising: (1) a first end segment configured for insertion into the lumen of a first graft piece; (2) a second end segment configured for insertion into the lumen of a second graft piece; and (3) a middle segment configured to maintain the graft piece ends in a spaced apart relationship; wherein each of the first and second end segments defines a groove or one or more rings for receiving a clip, clamp, tie filament or the like for attachment of the segments to the first and second graft pieces. In certain embodiments, the groove or ring is configured to receive a purse-string suture.

In certain embodiments, the lumen of the connector device is substantially straight. In other embodiments, the connector is not straight.

As noted above, one advantage of the invention is that use of a graft having a tapered or stepped down distal end enables a reduction in the size of the incision, which is expected to decrease the amount of endothelial injury and dampen the cascade leading to NIH. Furthermore, in embodiments including a cuff on the graft, a purse-string suture anastomosis can exclude the injured endothelium from the intravascular portion of the anastomosis. The cuff can also prevent slipping or dislodgment of the purse-string anastomosis which could result in obvious disastrous results. Decreasing the diameter of the graft at the exit also serves to dampen turbulent flow and make flow more laminar.

Though it is apparent that a tapering of the distal end will have an effect upon blood flow rates and pressures within an inventive graft, it is believed that inventive grafts will exhibit excellent long-term flow characteristics while also exhibiting other advantages apparent from the present description. Indeed, researchers conducting graft surveillance studies have reported that a large percentage of functioning AVGs have anastomotic or other stenoses greater than 50% of the original diameter. Other researchers have stated that the “critical stenosis for thrombosis is about a 70% decrease in diameter.” Hakim and Himmelfarb (1998). In an inventive graft having a 4 millimeter distal end that is stepped down from a 6 millimeter section, the diameter indeed decreases at the distal end, but only by 33%. In addition, in embodiments that are tapered at each end (i.e., at the distal end and the proximal end), it is believed that volumetric flow rate through the graft will not be significantly altered by having a stepped down distal end.

It is expected that resistance to flow resulting from having a tapered distal end will result in a decreased rate of flow within the portion of the graft having a larger diameter. It has been reported that a flow greater than 800 ml per min is critical to prevent thrombosis. Hakim and Himmelfarb (1998). It is believed, for reasons stated above, that an inventive graft would remain patent for an acceptable period of time with an adequate blood pressure and arterial inflow. Although there is some possibility that thrombosis in the larger diameter portion of an inventive graft resulting from a reduced rate of flow may be a concern in the use of inventive grafts, it is expected that an increased tendency toward thrombosis, if present, can be addressed by using anticoagulants and/or antiplatelet drugs.

In addition, Lumsden commented that the length of a segment having a 4 millimeter diameter on a stepped graft at the arterial anastomosis might adversely affect patency if greater than 2 centimeters. The difference, if statistically significant, was not outlined; but this would be consistent with Poiseuille's Law and with Hakim and Himmelfarb's findings regarding flow rate. In applying the Hagan-Poiseuille Law:

$$Q = \frac{Π {R}^{4} Δ P}{8 η L}$$

when other variables remain unchanged, as the radius (R) of a tube decreases, the volume flow rate (Q) decreases, and not linearly. As the length (L) (of the narrowed area) increases, the flow rate (Q) also decreases. Lumsden's findings would be expected to apply similarly to the distal end. Therefore, one preferred embodiment of the invention includes a stepped-down distal end portion having dimensions (i.e., radius and length of stepped-down portion) that do not significantly increase the risk of thrombosis of the graft. In one preferred embodiment, the stepped-down distal end portion has a length of no greater than about 3 centimeters. In another embodiment, the stepped-down distal end portion has a length of no greater than about 2 centimeters. In another embodiment, the stepped-down distal end portion has a length of no greater than about 1 centimeter.

As mentioned above, a widespread belief and a current trend is to implant a graft having a relatively large venous anastomosis. The belief and trend is thought to result from the notion that distal end graft occlusion would be delayed in a graft having a large venous anastomosis because a greater degree of stenosis would be required to narrow the venous anastomosis beyond the critical size necessary for patency. A person of ordinary skill in the art would find no motivation in the prior art to use an AVG having a smaller distal end and, indeed, a person of ordinary skill in the art would conclude that a smaller anastomosis would lead to an accelerated point of critical stenosis and occlusion. It is believed that a smaller anastomosis in the classical manner (end to side) would result in the development of NIH and a more abrupt “critical stenosis” with thrombosis; however, the present invention provides unique devices and methods that enable the placement of an AVG having a stepped down or tapered distal end.

All references, including publications, patents, and patent applications, cited or listed in this specification are herein incorporated by reference as if each individual reference were specifically and individually indicated to be incorporated by reference and set forth in its entirety herein. Further, any theory, proposed mechanism of operation, or finding stated herein is meant to further enhance understanding of the present invention, and is not intended to in any way limit the present invention to such theory, proposed mechanism of operation, or finding. While the invention has been illustrated and described in detail in the drawings and foregoing description, the same is to be considered as illustrative and not restrictive in character, it being understood that only the preferred embodiments have been shown and described and that all changes, equivalents, and modifications that come within the spirit of the invention defined by following claims are desired to be protected.

What is claimed is:

1. A vascular graft defining a lumen for passage of blood, comprising:
a distal end adapted for placement in a blood-receiving blood vessel through a wall of the vessel, the distal end defining a first orifice having a first diameter;
a proximal end adapted for attachment to an artery site;
a first tubular section between the distal and proximal ends, the first tubular section defining a portion of the lumen having a second, generally constant diameter greater than the first diameter; and
a first taper section between said first tubular section and said distal end, and having a generally gradually decreasing diameter along the first taper section.

2. The graft in accordance with claim 1, wherein the second diameter is at least about 20% greater than the first diameter.

3. The graft in accordance with claim 1, wherein the second diameter is at least about 30% greater than the first diameter.

4. The graft in accordance with claim 1, wherein the first diameter is no greater than about 6 millimeters.

5. The graft in accordance with claim 1, wherein the first diameter is no greater than about 5 millimeters.

6. The graft in accordance with claim 1, wherein the first diameter is from about 3.5 to about 4.5 millimeters and wherein the second diameter is from about 5.5 to about 7.0 millimeters.

7. The graft in accordance with claim 1, further comprising a second tubular section between said distal end and said first taper section, said second tubular section defining a portion of the lumen having a generally constant diameter corresponding to the first diameter.

8. The graft in accordance with claim 7, wherein the longitudinal length of the first taper section is less than about 2 centimeters.

9. The graft in accordance with claim 7, wherein the first taper section is positioned from about 1 to about 10 centimeters from said distal end.

10. The graft in accordance with claim 1, wherein the longitudinal length of the first taper section is from about 2 to about 10 centimeters.

11. The graft in accordance with claim 1, wherein the first taper section extends from said distal end to a longitudinal point at least about 4 centimeters from said distal end.

12. The graft in accordance with claim 1, wherein the first taper section extends from a longitudinal point from about 4 to about 15 centimeters from said distal end to said distal end.

13. The graft in accordance with claim 1, wherein said proximal end defines a second orifice having a third diameter less than the second diameter; and said graft further comprising a second taper section between said proximal end and said first tubular section, the second taper section having a generally evenly increasing diameter.

14. The graft in accordance with claim 13, further comprising a third tubular section between said proximal end and said second taper section, wherein said third tubular section defines a portion of the lumen having a generally constant diameter corresponding to the third diameter.

15. The graft in accordance with claim 14, wherein the second diameter is at least about 20% greater than the third diameter.

16. The graft in accordance with claim 14, wherein the third diameter is no greater than about 6 millimeters.

17. The graft in accordance with claim 14, wherein the first diameter is from about 3.5 to about 4.5 millimeters; wherein the second diameter is from about 5.5 to about 6.5 millimeters; and wherein the third diameter is from about 3.5 to about 4.5 millimeters.

18. The graft in accordance with claim 13, wherein said first tubular section has a length that is at least about 60% of the length of the graft.

19. The graft in accordance with claim 13, wherein said first tubular section has a length that is at least about 70% of the length of the graft.

20. The graft in accordance with claim 13, wherein said first tubular section has a length that is at least about 80% of the length of the graft.

21. The graft in accordance with claim 13, wherein said first tubular section has a length that is at least about 95% of the length of the graft.

22. The graft in accordance with claim 1, further comprising a cuff affixed to the outer surface of the graft at least about 1 centimeter from said distal end, the cuff defining a groove configured to receive a purse-string suture in the blood vessel wall for anastomosis.

23. The graft in accordance with claim 22, wherein the groove lies generally on a plane that is at an angle of about 45 degrees to a longitudinal axis of the graft.

24. The graft in accordance with claim 22, wherein said cuff is positioned from about 1 to about 10 centimeters from said distal end.

25. The graft in accordance with claim 22, wherein said cuff is positioned from about 1 to about 5 centimeters from said distal end.

26. The graft in accordance with claim 22, wherein said cuff comprises:
a tubular member defining a lumen sized to engage an external surface of said graft; and
first and second ridges extending around said member and defining the groove therebetween.

27. The graft in accordance with claim 22, wherein said cuff comprises:
a first ring member affixed to said graft; and
a second ring member affixed to said graft;
wherein the first and second ring members define the groove therebetween.

28. The graft in accordance with claim 27, wherein the first ring is positioned from about 1.5 to about 2 centimeters from the distal end and wherein the second ring is positioned from about 2 to about 2.5 centimeters from the distal end.

29. The graft in accordance with claim 27, wherein the first ring is positioned about 1.75 centimeters from the distal end and wherein the second ring is positioned about 2.25 centimeters from the distal end.

30. A method, comprising:
making an incision in the wall of a preselected target blood vessel;
providing a vascular graft defining a lumen for passage of blood, the graft including:
a distal end adapted for placement in the blood vessel through the incision and defining a first orifice having a first diameter;
a proximal end adapted for attachment to an artery;
a first tubular section between the distal and proximal ends, the first tubular section defining a portion of the lumen having a second, generally constant diameter greater than the first diameter; and
a first taper section between said first tubular section and said distal end, and having a generally gradually decreasing diameter along the first taper section;
inserting the distal end through the incision into the blood vessel such that the distal end passes to a point downstream of the incision;
securing the graft to the blood vessel wall; and
anastomosing the proximal end of the graft to a preselected target artery site.

31. The method in accordance with claim 30, wherein the graft is secured to the blood vessel wall using a purse-string suture.

32. The method in accordance with claim 31, wherein the purse-string suture is inserted in a wall of the target blood vessel prior to making the incision.

33. The method in accordance with claim 30, wherein said inserting comprises inserting the distal end through the incision into the blood vessel such that the distal end passes to a point at least about 1 centimeter downstream of the incision.

34. The method in accordance with claim 30, wherein the blood vessel has a diameter of less than about 1.5 centimeters.

35. The method in accordance with claim 30, wherein the blood vessel has a diameter of no greater than about 1.4 centimeters.

36. The method in accordance with claim 30, wherein the blood vessel has a diameter of no greater than about 1.3 centimeters.

37. The method in accordance with claim 30, wherein the blood vessel has a diameter of up to about 3 centimeters.

38. The method in accordance with claim 30,
wherein the graft comprises a cuff affixed to the outer surface of the graft at least about 1 centimeter from said distal end, the cuff defining a groove configured to receive a purse-string suture for anastomosis;
wherein said inserting comprises inserting the distal end of the graft through the incision into the blood vessel such that the groove is positioned to receive the blood vessel wall; and
wherein said securing comprises securing the blood vessel wall to the cuff.

39. The method in accordance with claim 38, wherein the blood vessel wall is secured to the cuff using a purse-string suture.

40. The method in accordance with claim 39, wherein the purse-string suture is inserted in a wall of the target blood vessel prior to making the incision and wherein said securing comprises drawing the purse-string suture into the groove.

41. The graft in accordance with claim 1, wherein said graft comprises an externally detectable marking.

42. The graft in accordance with claim 1, wherein said cuff comprises an externally detectable marking.

43. The graft in accordance with claim 41, wherein the externally detectable marking is a radiopaque marking.

44. The graft in accordance with claim 41, wherein the externally detectable marking includes a material having a density that is different than the density of a patient's body.

45. The graft in accordance with claim 41, wherein the externally detectable marking is a paint or coating including an externally detectable material.

46. The graft in accordance with claim 41, wherein at least a portion of said cuff comprises an externally detectable material.

47. The method in accordance with claim 30, wherein the graft comprises an externally detectable marking.

48. The method in accordance with claim 38, wherein the cuff comprises an externally detectable marking.

49. A method comprising:
anastomosing a first end of a vascular graft to a first blood vessel;
anastomosing a second end of the graft to a second blood vessel;
removing a medial portion of the graft by clipping the graft at two locations between the first end and the second end, thereby providing a first graft piece comprising the first end and a second graft piece comprising the second end;
providing a generally tubular connector device defining a lumen therethrough, the device having a first end segment configured for insertion into the lumen of the first graft piece; and a second end segment configured for insertion into the lumen of the second graft piece; and
connecting the first and second graft pieces to the first and second portions of the connector device.

50. The method according to claim 49, wherein the connector device further includes a middle segment configured to maintain the graft piece ends in a spaced apart relationship.

51. The method in accordance with claim 49, wherein one or both of the first and second end segments defines a groove for receiving a clip, clamp or tie filament for attachment to one or both of the first and second graft pieces.

52. The method in accordance with claim 49, wherein one or both of the first and second end segments is coated with an adhesive composition for attachment to one or both of the first and second graft pieces.

53. The method in accordance with claim 49, wherein one or both of the first and second end segments includes two rings defining a groove therebetween for receiving a clip, clamp or tie filament for attachment to one or both of the first and second graft pieces.

54. The method in accordance with claim 49, wherein one or both of the first and second end segments includes a ring for receiving a clip, clamp or tie filament for attachment to one or both of the first and second graft pieces.

55. The method in accordance with claim 49, wherein one or both of the first and second end segments defines a tapered end portion.

56. The method in accordance with any of claims 49-55, wherein said connecting comprises:
inserting the first portion into the lumen of the first graft piece;
securing the second portion to the second graft piece;
inserting the second portion into the lumen of the second graft piece; and
securing the second portion to the second graft piece.

57. A generally tubular connector device defining a lumen therethrough, the device comprising:
a first end segment configured for insertion into the lumen of a first graft piece;
a second end segment configured for insertion into the lumen of a second graft piece; and
a middle segment configured to maintain the graft piece ends in a spaced apart relationship.

58. The device in accordance with claim 57 wherein one or both of the first and second end segments is coated with an adhesive composition to attach the segments to the first and second graft pieces

59. The device in accordance with claim 57 wherein one or both of the first and second end segments defines a groove for receiving a clip, clamp or tie filament for attachment to one or both of the first and second graft pieces.

60. The device in accordance with claim 57 wherein one or both of the first and second end segments includes two rings defining a groove therebetween for receiving a clip, clamp or tie filament for attachment to one or both of the first and second graft pieces.

61. The device in accordance with claim 57 wherein one or both of the first and second end segments includes a ring for receiving a clip, clamp or tie filament for attachment to one or both of the first and second graft pieces.

62. The device in accordance with claim 57 wherein one or both of the first and second end segments defines a tapered end portion.

63. A generally tubular connector device defining a lumen therethrough, the device comprising:
a first end segment configured for insertion into the lumen of a first graft piece; and
a second end segment configured for insertion into the lumen of a second graft piece;
wherein each of the first and second end segments defines a groove for receiving a suture for attachment of the segments to the first and second graft pieces.

64. The device in accordance with any of claims 57-63, wherein the lumen of the connector device is substantially straight.
